(19) United States
(12) Patent Application Publication
(10) Pub. No.: US 2003/0208121 A1
(43) Pub. Date: Nov. 6, 2003

(54) Manipulator for a closed magnetic resonance tomograph

(76) Inventors: Horst Baltschun, Rulzheim (DE); Jorg Vagner, Eggenstein-Leopoldshafen (DE); Judith Bast, Worth State (DE); Holger Frenzel, Bruchsal (DE); Siegfried Schonherr, Eggenstein-Leopoldshafen (DE); Werner Alois Kaiser, St. Augustin (DE)

Correspondence Address: KLAUS J. BACH & ASSOCIATES, PATENTS AND TRADEMARKS, 4407 TWIN OAKS LANE, MURRYSVILLE, PA, 15668, US

(21) Appl. No.: 10/444,841

(22) Filed: May 23, 2003

Related U.S. Application Data
(63) Continuation-in-part of application No. PCT/EP01/14535, filed on Dec. 11, 2001.

(30) Foreign Application Priority Data
Dec. 20, 2000 (DE) 100 63 566.0

(51) Int. Cl. A61B 5/05

(52) U.S. Cl. 600/410

(57) ABSTRACT

In a manipulator for use in a closed magnetic resonance tomograph with an MRT channel extending therethrough for receiving a patient's rest supporting a female patient laying on her stomach during mammography, a support arm having a front end with a receiving housing for accommodating various instruments for the treatment of the patient is provided, which support arm is movable into the channel together with the patients rest and which is positionable relative to the patients mamma steplessly in every direction and operable in a position adjacent to the mamma for any desired treatment of the mamma.

This is a Continuation-In-Part application of international application PCT/EP01/14535 filed Dec. 11, 2001 and claiming the priority of German application 100 63 566.0 filed Dec. 12, 2001.

### BACKGROUND OF THE INVENTION

The invention relates to a manipulator for use in a closed magnetic resonance tomograph, MRT, which includes a channel for receiving a patients rest for female patients disposed on their stomach during mammography. The manipulator includes a support arm with a housing supporting instruments, which can assume any desired position relative to the mamma of the patient in a stepless manner. The patient rest can be moved together with the patient parallel to the longitudinal axis of the channel into the channel and the instruments are operable in the channel for surgical procedures on the mamma. In a closed MRT—in contrast to an open MRT—there are no side openings in the channel.

With the known closed MRT, the MRT-based mammography with follow-up treatments such as a biopsy, and, possibly a subsequent therapy requires a plurality of steps whereby the patient must be repeatedly moved out of and back into the channel. A reason herefor is that, with a closed MRT, the surgical treatment steps can be performed on a patient only outside the apparatus. This however is time consuming and provides for high physical and pyschological tensions for the patient and, furthermore, results possibly in a position change of the mamma, particularly during extended procedures. The high time requirements also render the therapy relatively expensive.

DE 196 39 975 discloses an MRT with a channel extending therethrough for the reception of a patient, but without any instruments for the treatment of the patient.

EP A 0 534 607 discloses an MRT with annular magnets within which a patient is supported and between which a mechanical device is provided at a side of the apparatus, which mechanical device is provided at a side of the apparatus. The device is not described in detail except that it provides for heat treatment of tumors of a patient by means of laser energy. No previous biopsy is considered in connection with the MRT according to EP-A-0 534 607. For performing a biopsy, the patient would have to be removed from the MRT. There is no indication or hint that a biopsy could be performed on a patient while in the apparatus.

DE 196 26 286 A1 and DE 295 21 366 U1 further disclose breast coils for use with an open MRT, which are provided at their sides with biopsy apparatus movable with various degrees of freedom but which are accessible only from without the MRT. With this apparatus, MRT supported biopsy can be performed only outside of a closed MRT so that the patient has to be moved out of the measurement positions in the channel of the MRT whenever a biopsy must be performed. A biopsy in the MRT within view or the surgeon could therefore not be performed with this apparatus.

U.S. Pat. No. 5,443,068 discloses a system for an MRT supported but not invasive treatment of tumors by local ultrasonic heating. The system consists of a closed MRT (with a channel extending therethrough) for the introduction from one end thereof of a patients rest, into which a manipulator with an ultrasonic transducer is integrated directly below the patient support surface. The manipulator includes a pan-like support arm for the ultrasound transducer which is laterally movable along all three spatial axes and radiates and focuses always upwardly, and for carrying the coupling medium necessary for the transmission of the ultrasonic waves. For the transmission of the ultrasound waves to the patients body, a membrane is disposed on the coupling medium on which the patients body, preferably the mamma, is disposed and to the shape of which the membrane adapts. Lateral movability of the ultrasound transducer within the coupling medium along each of the three spatial axes is achieved by electric motors via jointed shafts and transmissions separately for each spatial axis. The electric motors are arranged outside the MR magnetic field and do not influence the MR magnetic field.

DE 198 18 785 A1 discloses a manipulator for a closed MRT with a channel extending therethrough for the reception of a patients rest for female patients laying on their belly during mammography. It includes a support arm and a housing at the end of the support arm. The housing includes the instruments for the treatment of the patient and can be moved into the channel together with the patient. The instruments can be positioned relatively to the mamma independently in any direction and can be operated in any given position relative to the mamma. The support arm is mounted to a pivot arm, which is supported from a horizontally guided carriage on a frame covering the whole cross-section of the MRT channel. By pivoting the pivot arm, the movement of the carriage and the displacement of the manipulator parallel to the longitudinal axis of the channel, the end of the support arm can be moved relative to the mamma in the longitudinal direction of the channel in a stepless fashion in any direction. For a horizontal lowering and raising of the pivot arm and also for its pivoting however the carriage needs to be moved.

Based on this state of the art, it is the object of the present invention to provide an improved manipulator for an MRT which is easier to operate and to handle. It also should be relatively small and rigid and its motion during movement of the manipulator end relative to the mamma should be easy to control for improved operational safety. The position of the manipulator relative to the patient the patient rest and the MRT should remain the same over the whole procedure.

### SUMMARY OF THE INVENTION

In a manipulator for use in a closed magnetic resonance tomograph with an MRT channel extending therethrough for receiving a patient's rest supporting a female patient laying on her stomach during mammography, a support arm having a front end with a receiving housing for accommodating various instruments for the treatment of the patient is provided, which support arm is movable into the channel together with the patients rest and which can be positioned relative to the patients mamma steplessly in ever direction and operated in a position adjacent to the mamma for any desired treatment of the mamma.

Below, the invention will be described in detail on the basis of the accompanying drawings.

### BRIEF DESCRIPTION OF THE DRAWINGS

FIGS. 1a and 1b are top views showing the manipulator in different angular pivot positions,

FIG. 2 is a sectional side view of the manipulator,

FIG. 3 shows an MRT in cross-section with the manipulator pivoted to one side,

FIGS. 4 and 5 show cross-sectional view of the reception housing at the front end of the manipulator arm shown from the side and from the top, and FIGS. 6a-h show the front end of the manipulator during different procedure steps of the biopsy procedure.

### DETAILED DESCRIPTION OF AN EMBODIMENT

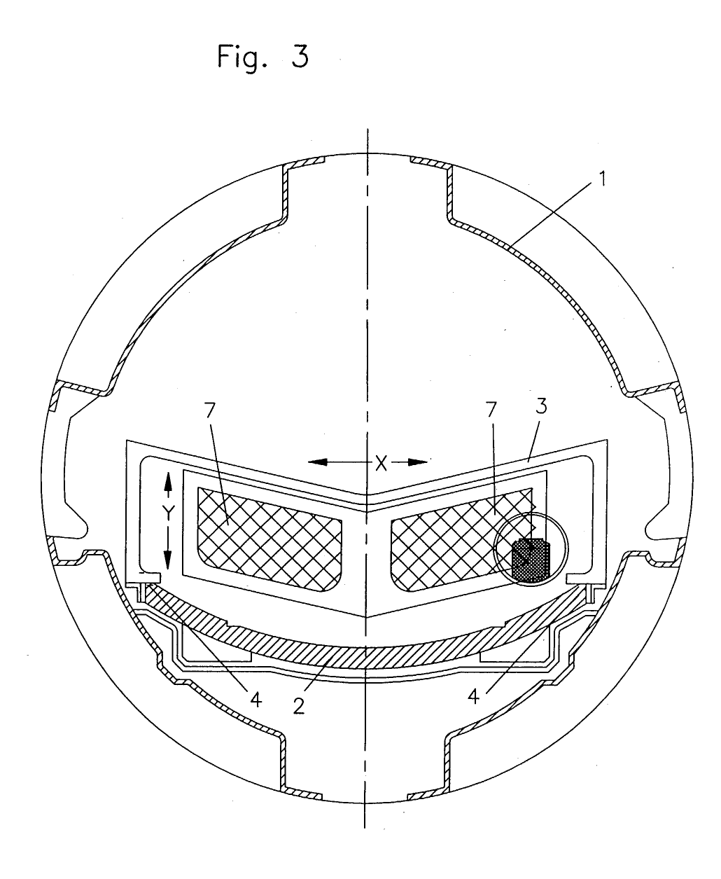

The manipulator shown in the figures is for use in a closed MRT with a channel 1 extending therethrough. The channel 1 is indicated in FIG. 3. FIG. 3 shows also a patient rest 3 for female patients suitable for mammography and possibly further treatment disposed in the channel 1 which patients rest can be moved into and out of the channel from one end thereof together with a table 2 supporting the patients rest 3 and also a manipulator. The manipulator and the patients rest 3 are disposed as separate units on the table 2 one behind the other so as to be movable relative to each other in the longitudinal direction of the table 2. They are supported at the longitudinal side edges of the table which form guide tracks 4. Of course, the whole manipulator as well as the patients rest 3 and the table 2 consist of an MRT compatible material.

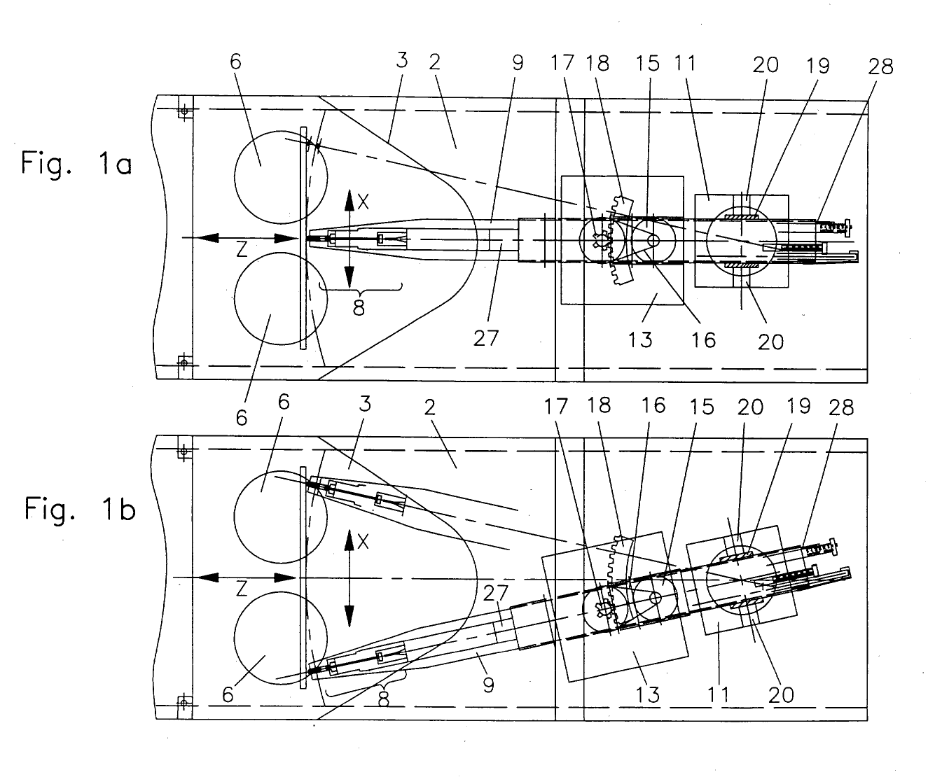

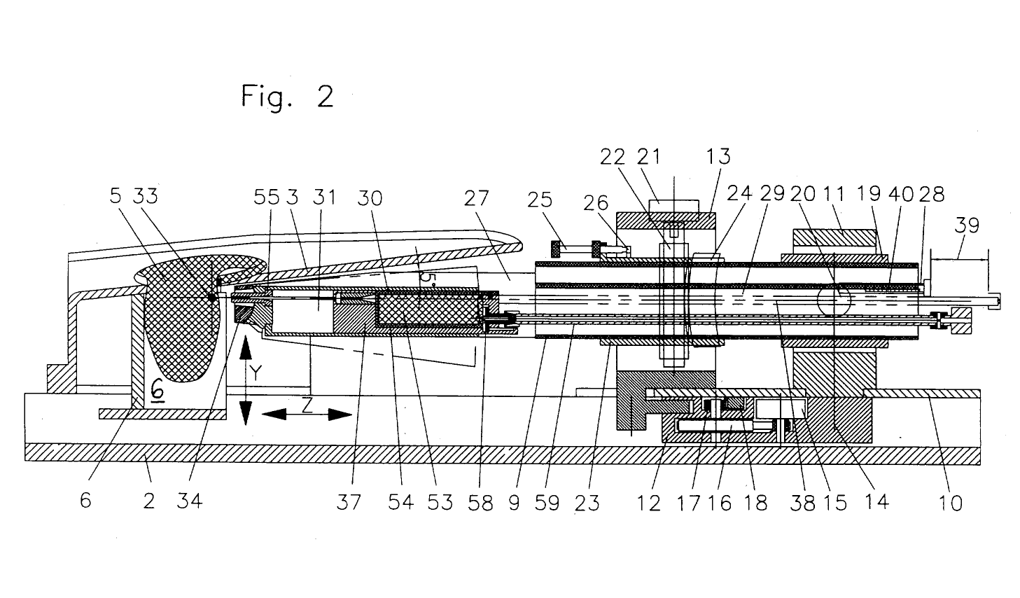

Below, the horizontal direction normal to longitudinal direction of the table 2 and the channel 1 (see FIGS. 1a, 1b, and 3) is defined as the x-axis and the vertical direction normal to the x-axis (see FIGS. 2 and 3) is defined as the y-axis. The axis which extends normal to the x and y-axis, that is parallel to the longitudinal direction of the table 2 and the channel 1, is designated as the z-axis (See FIGS. 1a, 1b, and 2).

On the patients rest, a female patient is disposed front side down (the patient is not shown in FIG. 2, but only the mamma 5), the mammas 5 extending downwardly through two openings 6. The different positions of the mammas (breasts) possible by anatomical differences can be accommodated by the two windows 7 indicated in FIG. 3 which determine the operating areas for the manipulator in the vertical plane normal to the longitudinal direction of the table 2 or the channel 1 (x/yplane). The mamma 5 to be treated is fixed there in the longitudinal direction by pressure plates 6 or/and a frame and positioned in the iso-center of the MRT by movement of the patient rest 3 on the table 2. Subsequently, the manipulator is moved on the table 2 in z-direction toward the mamma 5 to be examined such that its front end 8 is disposed adjacent the mamma as shown in FIGS. 1a and 1b and also in FIG. 2. In, or respectively at, the front end 8, the manipulator carries instruments as required for the treatment (see FIGS. 2 and 4 to 6).

The manipulator consists essentially of two components: a kardanically acting pivot arrangement for the support arm 9 and the manipulator arm consisting of the support arm 9 and the front end 8, which is mounted to the support arm 9 and comprises a housing receiving the medical instruments.

As shown in FIG. 2, the kardanically operating pivot arrangement for the support tube 9 is disposed on a base plate 10, which is supported on the table 1 so as to be movable in the z-direction.

A bore in the base plate 10 serves as a bearing for defining a common horizontal pivot direction (approximately in x-direction in the center position of the manipulator) for rigidly joined design components comprising a pivot housing 11, drive blocks 12, drive housings 13 and consequently also for the support arm 9, about the axis 14. The pivot movement of the manipulator is initiated by an MRT compatible motor, preferably a piezo motor 15 which drives a pinion 17 by way of a belt or gear transmission 16. The pinion 17 is in engagement with a. curved gear segment 18 firmly connected to the base plate 10, so that the drive block 12 can be moved relative to the base plate 10 about the axis 14.

The support arm 9 of the manipulator which,. in its neutral center position as shown in FIGS. 1a and 2, is. oriented along the z-axis, extends through openings in the pivot housing 11 and the drive housing 13 (see FIG. 2) provided specifically in these components. The reception sleeve 19 for the support arm 9 includes in the area of the pivot housing 11 horizontally projecting bolts 20, which extend normal to the sleeve axis in opposite directions and which are supported in the pivot housing 11 for permitting a vertical pivot movement of the pivot arm 9 (essentially in y-direction when in the center position). As with the earlier-described horizontal pivot movement, the vertical pivot movement is obtained by way of an MRT compatible motor, preferably a piezo motor 21, which is arranged in the drive housing 13 and drives worm gear 22 engaging a curved gear section 24 connected to a support sleeve 23 firmly mounted to the support arm 8. As a result, a relative movement between the drive housing 13 and the support arm 9 about the bolt 20 can be initiated. This area is shown in detail only in FIG. 2, whereas FIGS. 1a and 1b show the positions of the manipulator arm 9 in the pivot housing 11 and the drive housing 13 schematically.

As described earlier, the manipulator can be moved in the z-direction by manually moving the base plate 10 on the table 2 relative to the patients rest. In addition, the manipulator position can be fine-adjusted along the support arm axis (corresponding to the z-direction) from outside the MRT channel by means of a manual set screw 25 which extends into a threaded opening in the support sleeve 23 and which engages a stop 26 disposed on the support arm 9. Alternative to the manual set screw 25, the set screw may be provided with an MRT compatible piezo motor for computer-controlled positioning in the MRT.

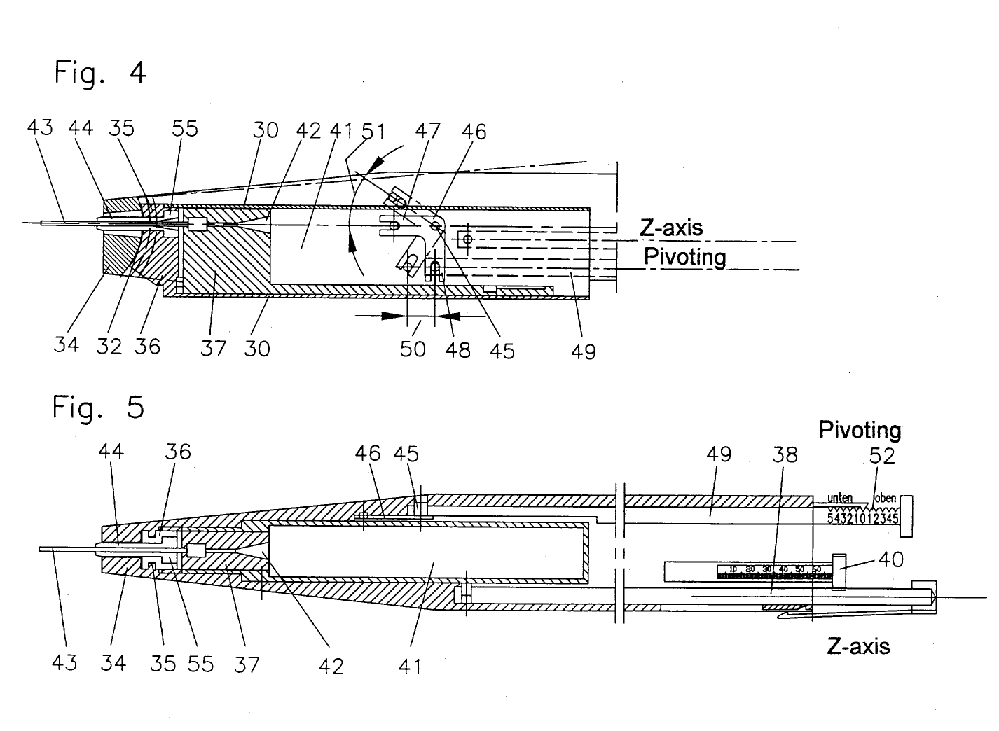

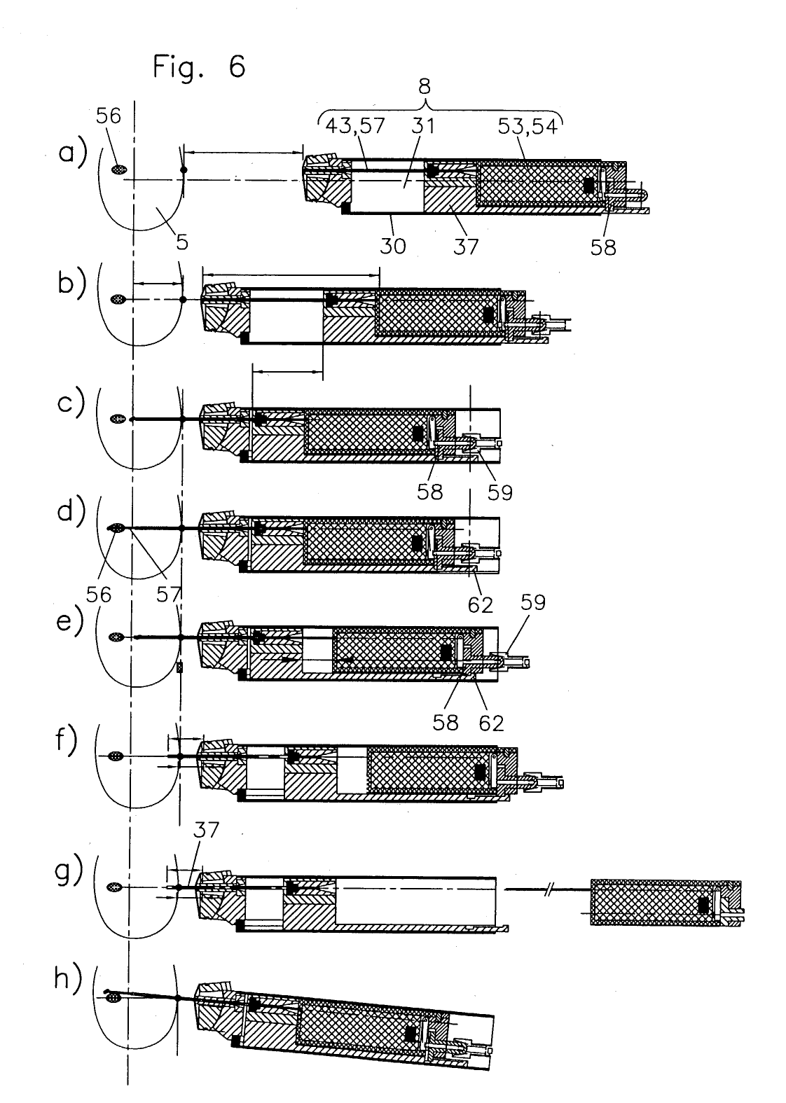

The second component group of the manipulator comprises the manipulator arm, which will be described below on the basis of FIG. 2 and the detail sectional views of FIGS. 4 and 5.

The manipulator consists of a support arm 9 having a front end 8 with a receptacle for medical instruments. As described earlier, the manipulator arm together with the support arm 9, is disposed in the support sleeve 23 and the reception sleeve 19 so as to be axially movable and also pivotable therein. In the area between the support sleeve 23 and the front end 8, the support arm is provided at its top and bottom with elongated openings 27 so as to permit an exchange of surgical instruments as will be described later.

Furthermore, the support arm has an instrument channel 29 between the proximal end 28 and the elongated opening 27. This instrument channel 29 is provided for the insertion and the operation of the surgical instruments at the front end 8 of the manipulator arm from the proximal end 28.

Another important component of the manipulator arm is the instrument receiving compartment 20, which is inserted into the elongated opening 27 and has an interior space 31. The instrument receiving compartment 30 is pivotable vertically and—after rotation of the support tube 9 in the support sleeve 23 and the reception sleeve 19 by 90° —also horizontally around the circle segment 32 (FIG. 4) forming a friction bearing surface and about the invariable point 33. The circle segment 32 is formed by the backside of the distal end piece 34 of the manipulator and, with regard to the position shown in FIGS. 4 and 5, a vertical T-guide structure 35, in which the instrument receiving compartment 30 is slidingly supported by a front structure 36 having a correspondingly formed counter structure.

In the interior space 31 of the instrument receiving compartment 30, there is the receiving housing 37, which is movable therein in the z-direction (with respect to the horizontal center position) and into which the instruments necessary for treatment are inserted and where they can be exchanged. The receiving housing 37 can be moved normally by means of a push rod 38 from the proximal end 28 of the manipulator arm. The stroke 39 of the push rod 38 is limited by an adjustable stop 40 as shown in FIG. 2. The receiving housing 37 further includes at its proximal end a recess 41 for the insertion of a surgical instrument which, at the distal end, is received in a conical bore 42 in the receiving housing 37 and leads to a trokar 43 connected to the receiving housing 37. The trokar 43 is additionally guided at the front end 36 of the instrument receiving compartment 30 by a bearing sleeve 44, which extends through a bore in the distal end piece 34 of the manipulator.

In the receiving housing 37, an instrument 53 is inserted into an instrument adapter 54 shown in FIG. 2, and is axially movable within the instrument adapter 54 in the recess 41. In an actual application in the MRT mammography, the instruments inserted are a biopsy apparatus for the retrieval of a tissue sample and the applicator of a kryo or laser apparatus for a possible subsequent kryo therapy or laser surgery. To this end, instrument adapters 54 suitable for the particular instrument are utilized the particular instrument 5 being adapted to the recess 41 of the receiving housing 37.

The instrument adapter 54 can be locked to the instrument 53 in different positions by means of a coupling structure in the form of a track disc 58 selectively extending into the receiving housing 37 and the rotatable guide track disc 58 in the and, after release of the coupling arrangement, is removable from the receiving housing 37 in proximal direction.

A rod 59 is connected to the guide track disc 58 by means of a joint and extends to the proximal end 28 of the manipulator so that the instrument adapter 54 with the instrument 53 can be pulled and removed from the proximal end and again inserted. The function of the track disc 58 will be described in detail in connection with the FIGS. 6a to 6b.

If the instruments inserted must be protected in the reception housing in a sterile manner from contaminations by the trokar 43, a sterile sleeve 55 is inserted as protective component at the front structure 36 of the instrument receiving compartment 30 ahead of the trokar. The trokar 43 extends through the sterile sleeve 55 when the reception housing 37 is moved forward.

The receiving housing 37 is pivoted together with the instrument receiving compartment 30 about the invariable point 33, which is disposed outside the front end 8 (see FIG. 2) and at the point where the instrument enters the patients tissue so that the instrument 53 and the trokar 43 can be pivoted but the tissue penetration point remains the same. As shown in FIGS. 4 and 5, a pivot lever 46 with two legs 47, 48, which extend normal to each other, is supported on a shaft 45 mounted on the support arm 9 at the side of the instrument receiving compartment 30. One of the legs, that is, the leg 47, movably engages the instrument receiving compartment 30 and the other leg 48 is connected to an operating rod 49. A stroke 50 of the operating rod 49 generated by axial movement thereof pivots the pivot lever 46 about the axis 45 so that the leg 47 is pivoted upwardly or downwardly and the instrument receiving compartment 30 movably connected thereto is also pivoted together with the receiving housing 37 disposed therein and the trokar 43. Pivoting about the angle 51 pivots the instrument receiving compartment 30, the pivot movement being controllable by a scale with engagement means 52 having a degree indication at the proximal end of the push rod 49. For a fan biopsy, the instrument reception chamber 30 is pivoted up and down, degree by degree, by means of the push rod 49. In order to be able to pivot the instrument reception chamber 30 to the left and to the right, the support tube 8 has to be rotated in the reception sleeve 19 and in the support sleeve 23 by 90°.

The actual procedure of the MRT based biopsy and the procedural steps occurring thereby in the manipulator, particularly in the instrument reception chamber 30 will be explained below on the basis of FIGS. 6a to 6h.

First, the mamma 5 of the patient laying on her stomach on the patient rest 3 in the MRT channel 1 is positioned in the isocenter of the MRT in the area of the windows 7 (see FIG. 3) received in the openings 6 and is fixed in position for example by fixing plates or a fixing frame or an anatomically formed shell with openings. Subsequently, the planigraphic images are taken in the conventional manner whereby the target coordinates of possible findings are recorded. If a biopsy is to be performed for a finding, the target tissue 56 is defined and the coordinates thereof are converted by a computer into control signals for the manipulator adjustment by means of piezo electric motors (in x, y, and z direction). The computation of the control signal takes into account corrections on the basis of the possible pivot angles of the manipulator arm which deviate from the z-direction as well as the distance between the front end 8 and the target tissue in the mamma 5, which varies with the pivot procedure in such a way that the advance movement of the trokar 43 and the instrument 53 into the mamma are adjustable independently of the pivot position.

FIG. 6 shows the front end 8 of the manipulator arm with the instrument receiving compartment 30 which has not been moved yet toward the mamma 5 for the biopsy to be performed.

The receiving housing 37 with the instrument 53 is disposed in the instrument adapter 54 in the recess 41 in a retracted position, that is, the trokar 43 is not extended. In this start-out position, the instrument adapter 54 is locked at the rear end of the receiving housing 37 by means of the guide track disc 58c. The instrument 53 in this case is a biopsy apparatus whose biopsy needle 57 is inserted into the trokar 43. Furthermore, in the FIGS. 6a to 6h, the target tissue 56 for the 20 biopsy in the mamma 5 is shown.

As shown in FIG. 6b, for the biopsy, the manipulator tip is moved toward the mamma 5 first manually, then by means of a set screw 25 (see FIG. 2).

When the target tissue location has been determined, the positions of the front end 8 is adjusted in the x and y directions and the trokar is then inserted into the tissue up to shortly ahead of the target tissue (FIG. 6c) . In this step, the trocar 43 is advanced together with the receiving housing 37 in the interior space 31 of the instrument receiving compartment 30 by moving the push rod 38 (see FIG. 5), which engages the receiving housing 37. The insertion depth of the trokar is controlled by an adjustable stop 61 (see FIG. 5) and by the z-axis adjustment by way of the set screw 25 possibly by a piezomotor.

In this position, a tissue sample is taken from the target tissue 56 by operating the biopsy apparatus (instrument 53) using the biopsy needle 57. The biopsy apparatus is actuated for example by rotating the guide track disc 58 by means of the rod 59 or by a Bowden cable or a pull cable in a rod 59, which, in that case, is tubular (see FIG. 2).

After a sample has been taken, the instrument adapter 54 with the biopsy apparatus in the recess 41 of the receiving housing 37 is pulled back by the rod 59, whereby the biopsy needle 57 with the tissue sample is pulled back into the trokar 43 (FIG. 6e). To this end, the lock of the instrument adapter 54 in the receiving housing 37 is released by a defined rotation of the guide track disc 58 by means of the rod 59. This retracting movement is limited by a proximal stop 62 for the guide track disc 58 in a corresponding angular position on the receiving housing 37.

As shown in FIG. 6f, the trokar 43 is retracted by backward movement of the receiving housing 37 in the interior space 31 of the instrument receiving compartment 30 via the push rod 38 to such an extent that the trokar tip remains still in the mamma to facilitate the insertion of other instruments for possibly necessary other treatment procedures.

After a further predetermined rotation of the guide track disc 58, the guide track disc 58 is moved off the proximal stop 62 whereby the biopsy apparatus together with the instrument adapter 54 is uncoupled from the receiving housing 37 and can be pulled out at the proximal end thereof. (FIG. 6g). Since the trokar 43 still extends into the mamma 5, another instrument 53 can easily be inserted for further treatment.

For further treatment, for example, a kryo- or laser applicator may be inserted in place of the biopsy apparatus. Such an applicator is introduced into the receiving housing 37 in the same way as the biopsy apparatus as the housing 37 is still positioned in accordance with the target coordinates of the manipulator. In this retained position, kryo- or laser energy is applied corresponding to the size of a tumor as determined.

FIG. 6h shows the principle of a fan biopsy. Herefor, the instrument receiving compartment 30 with the receiving housing 37 is pivoted by means of the pushrod 49 and the pivot lever 46 as shown in FIG. 4.

In another possible embodiment of manipulator, the operating mechanism for the positioning is not driven by a motor by way of the earlier described transfer elements but is operated fluidically by hydraulic or pneumatic operating elements, preferably a control cylinder, in connection with a closed control circuit.

What is claimed is:

1. A manipulator for use in a closed magnetic resonance tomograph, MRT, having an MRT channel extending therethrough for receiving a patient's rest supporting a female patient laying on her stomach during MR mammography, including a support arm having a front end, and a receiving housing disposed in said front end for accommodating instruments for the treatment of the patient, said support arm being movable into the channel parallel to the longitudinal axis thereof and being positionable relative to the mamma steplessly in every direction and operable in a position adjacent to the mamma, said support arm being further supported so as to be pivotable in a horizontal and in a vertical direction.

2. A manipulator according to claim 1, wherein said patients rest includes two openings for accommodating the mammas of the patient and a fixing structure is provided on the patients rest below the openings for positioning and fixing the mammas.

3. A manipulator according to claim 2, wherein said fixing structure includes a fixing frame and pressure plates engaging the mammas.

4. A manipulator according to claim 1, wherein a) the support arm is disposed in a pivot housing and a drive housing so as to be pivotable, MRT compatible piezo motors with motion transmissions providing for the pivoting and positioning of the support arm, b) the pivot housing and a drive housing are joined to form a unit which is supported on a base plate pivotally about a vertical axis, said base plate having a curved gear segment mounted thereon and said support arm including in said drive housing an MRT compatible motor with a transmission carrying a pinion in engagement with said curved gear segment for executing pivot movements of the support arm, said support arm further including in the area of the pivot housing bearing bolts extending horizontally in opposite directions by which the support arm is supported in a pivot housing and another piezo motor with a worm gear is disposed in the drive housing and operatively connected to the support arm by way of another curved gear segment.

5. A manipulator according to claim 1, wherein the manipulator and the patients rest are two separate units which are mounted on a table behind one another and which are movable together with the table into, and out of, the channel.

6. A manipulator according to claim 1, wherein said support arm of said manipulator has at its front end (distal end) top and bottom openings.

7. A manipulator according to claim 6, wherein, in the open area at the front end of the manipulator, there is an instrument receiving compartment which is supported on the manipulator by way of a circle segment so as to be pivotable about an invariant point in front of the front end of the manipulator, and the instrument receiving compartment includes slidably supported therein a receiving housing in which an instrument adapter is disposed to which instruments needed for examination or surgical procedures can be removably connected.

8. A manipulator according to claim 7, wherein a pivot lever with two legs extending at a right angle to each other is pivotally supported on a shaft extending from the support arm, one of the two legs engaging the receiving housing and the other leg being connected to an operating rod for pivoting the receiving housing about the invariant point, said operating rod having an operating end provided with a scale and with locking means.

9. A manipulator according to claim 1, wherein the distance of the front end of the manipulator from the patient rest along the longitudinal axis of the MRT channel is adjustable by means of a set screw operable by hand or by a piezo motor.
